(19) United States
(12) Patent Application Publication
(10) Pub. No.: US 2012/0004631 A9
(43) Pub. Date: Jan. 5, 2012

(54) WOUND PACKING MEMBERS

(75) Inventor: Edward Hartwell, York (GB)

(73) Assignee: Smith & Nephew Plc, London (GB)

(21) Appl. No.: 12/746,757

(22) PCT Filed: Nov. 17, 2008
(86) PCT No.: PCT/GB08/51075

§ 371 Date: Jun. 7, 2010

(30) Foreign Application Priority Data
Dec. 8, 2007 (GB) 0724044.3

(51) Int. Cl. A61F 13/15; D04B 1/22; B32B 38/00; D04C 1/00; B32B 37/06; B32B 37/12

(52) U.S. Cl. 604/367; 156/148; 156/60; 87/8; 66/170

(57) ABSTRACT

A method of making a three-dimensional wound packing member is described, the method comprising the steps of: taking material selected from at least one of the following forms comprising perforated sheet, net, woven, non-woven and knitted material; subjecting the at least one material to at least one forming process selected from the processes comprising rolling into tubes, braiding, knotting and knitting to form a three-dimensional and resilient structural unit member for packing into a wound.

The present invention relates to members for filling a volume of a wound particularly, though not exclusively, during topical negative pressure (TNP) therapy.

In recent years TNP therapy has become increasingly important in the field of improved treatment of wounds by making the healing thereof faster and more controlled.

The basic principle of TNP therapy is to create a closed cavity over the wound itself by means of a thin, flexible sealing film adhered to the patient's sound skin surrounding the wound; admitting one end of an aspirant conduit into the closed cavity, the conduit being sealed to the flexible film, for example; and connecting a distal end of the aspirant conduit to a vacuum source such as an electrically driven vacuum pump, for example, to create a pressure lower than the surrounding ambient atmospheric pressure within the wound cavity. As is known to the skilled person the lower pressure creates many beneficial therapeutic effects on the wound including increased blood flow to the wound and faster granulation of tissue, for example. There are very many variations on this basic principle of TNP therapy.

The types of wounds treated by TNP therapy generally range from quite small at about 5 cm² to very large traumatic wounds and burns of no particular maximum dimension. Such wounds may also have significant depth and therefore, significant volume. It is necessary to control the way in which a wound heals. For example, the wound should heal from the base up and close in from the edges desirably in a uniform manner. In particular it is desirable that the wound does not close over and form an occluded cavity in the flesh which is extremely undesirable from the patient's point of view as such form sites for infection.

To prevent the formation of occluded cavities during TNP therapy, the wound is usually packed with a filler which desirably has some resilience or “bounce” to resist the compressive forces created during TNP therapy by outside ambient atmospheric pressure bearing down on the wound due to the vacuum created in the wound cavity. The purpose of the filler is to keep the surrounding edges of the wound apart so that they cannot grow over and form a cavity. The filler may also perform the function of providing fluid flow channels between the wound and the filler in order to provide a uniform reduced pressure distribution over the surface area of the wound and to promote efficient aspiration of exudate fluids away from the wound surface and generally into a remote waste receptacle associated with the aspirant conduit.

As noted above there are very many variations on the basic TNP therapy principle and to illustrate how complex TNP therapy may be reference is made to the documents described below and which are of common ownership herewith.

In our co-pending International patent application, WO 2004/037334, apparatus, a wound dressing and a method for aspirating, irrigating and cleansing wounds are described. In very general terms, this invention describes the treatment of a wound by the application of topical negative pressure (TNP) therapy for aspirating the wound together with the further provision of additional fluid for irrigating and/or cleansing the wound, which fluid, comprising both wound exudates and irrigation fluid, is then drawn off by the aspiration means and circulated through means for separating the beneficial materials therein from deleterious materials. The materials which are beneficial to wound healing are recirculated through the wound dressing and those materials deleterious to wound healing are discarded to a waste collection bag or vessel.

In our co-pending International patent application, WO 2005/04670, apparatus, a wound dressing and a method for cleansing a wound using aspiration, irrigation and cleansing wounds are described. Again, in very general terms, the invention described in this document utilises similar apparatus to that in WO 2004/037334 with regard to the aspiration, irrigation and cleansing of the wound, however, it further includes the important additional step of providing heating means to control the temperature of that beneficial material being returned to the wound site/dressing so that it is at an optimum temperature, for example, to have the most efficacious therapeutic effect on the wound.

In our co-pending International patent application, WO 2005/105180, apparatus and a method for the aspiration, irrigation and/or cleansing of wounds are described. Again, in very general terms, this document describes similar apparatus to the two previously mentioned documents hereinabove but with the additional step of providing means for the supply and application of physiologically active agents to the wound site/dressing to promote wound healing.

The content of the above references is included herein by reference.

In spite of the self evident growing complexity of TNP therapy in general the field of wound fillers, which are a vital element in the therapy has changed or improved very little over the years that TNP therapy has been developing. Aside from complicated and expensive inflatable bags, most of the fillers in use are based either on foam or on cotton gauze. Foam fillers are usually cut with scissors to the required shape (of the wound) by a clinician. However both foams and gauzes have the disadvantage that the cell or pore size is often too large and often results in growing tissue growing into the cells and adhering the foam to the wound causing further damage and trauma to the wound and patient on removal. When gauze is used as a filler, however, clinicians are instructed to “fluff up” the gauze to increase its volume which can cause problems in that the actual form of the gauze as packed into wounds is very variable. A further disadvantage with both foams and gauze when cut to fit wounds is that of debris. Gauze and foam are particularly prone to shedding fibres and particles when cut and these inevitably find their way into the wound and become occluded therein which can lead to later infection.

It is an object of the present invention to overcome or mitigate some of the disadvantages of known wound fillers.

According to a first aspect of the present invention there is provided a method of making a three-dimensional wound packing member, the method comprising the steps of: taking material selected from at least one of the following forms comprising perforated sheet, net, woven, non-woven and knitted material; subjecting the at least one material to at least one forming process selected from the processes comprising rolling into tubes, braiding, knotting and knitting to form a three-dimensional and resilient structural unit member for packing into a wound.

The present invention may also comprise a further step of linking together a plurality of the structural units so formed into a chain, for example, by thread means. The chain so formed may then be used to pack a wound for TNP therapy. However, if the chain so formed provides too much volume then it may be reduced by removing a suitable number of chain units until of the appropriate overall desired volume is achieved. The linking thread means may, for example, be a monofilament thread such as Nylon (trade name), for example, so that cutting of the thread does not create any shedding or loose fibres.

The essence of the present invention is a method of making 3 dimensional wound packing means with variable pore size and compressibility from flat sheet material, for example. By taking sheet/net/woven/non-woven/knitted material and braiding, knotting or knitting and/or forming into tubes it is possible to create 3 dimensional structures with variable but controlled open volumes and densities. It is further possible to tie units of these structures so formed together into strings or chains with linking means to form larger structures. By changing the pore size, width of material between pores and thickness of the sheet/nets it is possible to vary the compressibility and pore size of the 3D structure. The user may vary the volume of the packer by adding suitable numbers of units of wound packing members together in a wound and in the case of strings may cut the appropriate volume of packing members at each linking entity. The packing members may be used as a general wound packer, or in conjunction with a sealing means and vacuum conduit as a packer for TNP therapy of wounds where it is essential that an even distribution of pressure takes place together with allowing for contraction of the wound, and intermittent contact of the wound with the packing member.

The selected material may be rolled, for example, into a tube or strand and that tube or strand with others may then be plaited, braided, knitted or woven for example, into a 3D structural member unit having controlled resilience and porosity. In an alternative structural wound packing member embodiment, a rolled tube of the selected material may then be further rolled along the axis of the tube in the manner of a ladies stocking, for example, so as to form a doughnut or ball shape depending upon the tightness of the starting rolled tube. A plurality of such doughnuts or balls formed in this manner may also be linked together to form a chain or string.

An important aspect of the present invention relating to linking a plurality of individual wound packing member units together is that when a specific number of units is cut from a chain of units then when these are removed from a wound at a time of dressing change then they are all removed together and there is no possibility of units remaining hidden in a deep wound, for example, as there is with a plurality of unconnected wound packing members.

An important advantage of the wound packing member units of the present invention is that they may be made from materials which do not naturally adhere to a wound surface such as, for example, polyurethane, polypropylene, ethylvinylacetate, silicone and the like. Further advantages of such material are that they do not shed fibres or particles when formed as extruded sheet or thermo-bonded net.

The packing member units so formed may be engineered to provide desired characteristics of porosity, compressibility and volume by controlling the degree of porosity/perforation in the initial starting sheet, for example, then controlling the degree of tightness with which the sheet is rolled, for example, and then controlling the degree of tightness with which a plurality of the rolled tubes are then braided or plaited or knitted together, for example.

Compressibility of the types of material contemplated as wound packing members according to the present invention may typically lie in the range from 0.01 to 0.5 kgf/cm², preferably from 0.025 to 0.050 kgf/cm² when measured at a compression deflection point of 40% according to DIN 53577.The materials remain freely porous to the flow of wound exudate at a compression pressure of 0.16 kgf/cm². Typically a material would deflect to about 50% to 90% of the relaxed volume at a pressure of 0.16 kgf/cm² and recover to about 90% of the original volume upon pressure release.

Integrity of the braided, plaited, knitted wound packing member units may be preserved by the additional step of adhesively bonding, heat sealing or tying with monofilament thread the ends of the units to prevent unravelling, for example.

It is intended that wound packing unit members are used as formed and not subdivided by cutting into smaller units which would to an extent defeat the object of eliminating shedding of particles into a wound.

According to a second aspect of the present invention there is provided a three-dimensional wound packing member unit when made by the first aspect of the present invention.

According to a third aspect of the present invention there is provided a kit comprising a plurality of three-dimensional wound packing member units according to the second aspect of the present invention linked together by a thread.

In order that the present invention may be more fully understood examples will now be described by way of illustration only with reference to the accompanying drawings, of which:

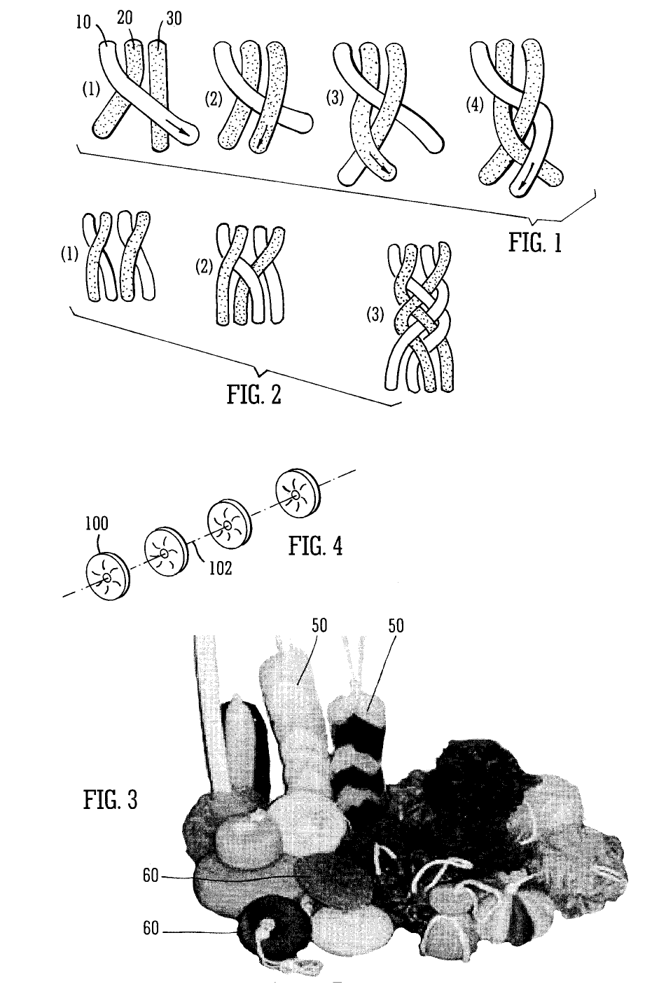

FIG. 1 shows a schematic of a plurality of rolled sheets of material being braided together;

FIG. 2 shows a schematic of another embodiment similar to FIG. 1;

FIG. 3 shows a photograph of various examples of wound packing members according to the present invention; and

FIG. 4 which shows a schematic view of a plurality of wound packing units joined together by a linking thread.

Referring now to the drawings and wherein in FIG. 1 shows stages in the braiding of three elements designated as 10, 12, 14 into a wound packing member unit. Each element is formed from a rolled sheet of about A4 size of perforated material such as, for example, perforated polyurethane film such as Elastogran SP806 (trade mark) of 100 g/m², 0.8 mm perforations at 2 mm pitch centres, having perforations therein, the shape, size and extent of perforations being predetermined so as to arrive at a desired overall porosity and resilience when a plurality of the elements are connected together.

FIG. 2 shows stages in a similar construction to that of FIG. 1 but the four rolled elements comprise Nylon (trade mark) net.

FIG. 3 is a photograph showing examples of embodiments of wound packing member units made according to the present invention. Wound packing members denoted at 50 are plaited structures and those at 60 are structures rolled like stockings.

FIG. 4 shows a plurality of rolled structures 100 which are joined together by a common linking monofilament Nylon thread 102. Although only four wound packing units are shown, a kit comprising a string of say 10 units may be provided and the clinician cutting off the appropriate number for the wound size concerned. This provides certainty at wound dressing change time that all old wound packing material has been removed from a wound because they are all linked together.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

1. A method of making a three-dimensional wound packing member, the method comprising the steps of: taking material selected from the group consisting of perforated sheet, net, woven, non-woven and knitted material; subjecting the at least one material to at least one forming process selected from the group consisting of rolling into tubes, braiding, plaiting, knotting and knitting, so as to form a three-dimensional and resilient structural wound packing unit member for packing into a wound characterised by a porosity level of the wound packing unit member being controlled by a degree of tightness of said forming process.

2. A method according to claim 1 wherein rolled tubes of the selected material are then formed into a resilient wound packing unit member by a forming process selected from the group consisting of braiding, plaiting, knotting and knitting, so as to form a resilient structural wound packing unit member.

3. A method according to claim 1 wherein the individual wound packing member units are further treated by a technique selected from the group consisting of adhesively bonding, heat sealing and mechanically fixing to preserve a structural integrity of each wound packing unit member.

4. A method according to claim 1 further comprising the step of linking together a plurality of the individual structural wound packing unit members so formed into a chain.

5. A method according to claim 4 further comprising the step of cutting the chain into an appropriate number of units to suit a specific wound to be treated.

6. A method according to claim 4 wherein the wound packing member units are linked by a thread.

7. A method according to claim 6 wherein the thread is a plastics material monofilament.

8. A method according to claim 1 wherein when the selected material is rolled into a tube, the porosity of the tube is partly controlled by the degree of tightness of rolling.

9. A method according to claim 6 wherein a porosity of the resulting wound packing member unit is partly controlled by the degree of tightness of a plaiting, braiding, knitting or knotting step.

10. A method according to claim 1 wherein materials comprising said wound packing member units do not adhere to growing tissue.

11. A method of making a three-dimensional wound packing member according to claim 1 wherein a tube is first formed by rolling of the selected material and them forming said tube into a doughnut shape by rolling of the tube along its axis.

12. A three-dimensional wound packing member unit comprising a material selected from the group consisting of perforated sheet, net, woven, non-woven and knitted material, wherein the material is processed so as to form a three-dimensional and resilient structural wound packing unit member, and wherein the wound packing member has a porosity level controlled by the processing.

13. (canceled)

14. (canceled)

15. (canceled)

16. (canceled)
